(19) United States
(12) Patent Application Publication
(10) Pub. No.: US 2002/0133225 A1
(43) Pub. Date: Sep. 19, 2002

(54) Methods and apparatuses for delivering a medical agent to a medical implant

(76) Inventor: Lucas S. Gordon, Sammamish, WA (US)

Correspondence Address: Brian P. Hopkins, Mintz, Levin, Cohn, Ferris,, Glovsky and Popeo, P.C., One Financial Center, Boston, MA, 02111, US

(21) Appl. No.: 10/081,770

(22) Filed: Feb. 22, 2002

Related U.S. Application Data
(60) Provisional application No. 60/275,682, filed on Mar. 13, 2001.

(51) Int. Cl. A61F 2/06

(52) U.S. Cl. 623/1.42

(57) ABSTRACT

A system and method for delivering a medical agent to a functional implant within a target tissue of an organism includes disposing a ferromagnetic, functional implant in a target tissue of an organism, the implant either having a magnetic field or being capable of magnetization, and introducing a medical agent carried by a magnetically sensitive carrier. The carrier is introduced into a blood flow of the organism upstream from the target tissue, the carrier and medical agent migrate via the blood flow to the target tissue, and the carrier and medical agent remain substantially localized around the implant as a result of the magnetic field. The implant may be magnetized via a permanent or electro magnet. In addition, the implant may be demagnetized by a degaussing device after the need for the medical agent is no longer required.

This application claims benefit under 35 U.S.C. §119(e) of U.S. Provisional Application No. 60/275,682, filed Mar. 13, 2001, the entire disclose of which is herein incorporated by reference.

### BACKGROUND OF THE INVENTION

1. Field of the Invention

The present invention pertains to prosthetic implants and devices that incorporate systems for local delivery of therapeutic and diagnostic agents to their location.

2. Background of the Prior Art

A variety of devices are implanted into patients to relieve a number of diseases and injuries. One such device is the stent. Stents are expandable, porous, metal or plastic tubes used to maintain an open lumen within a body passageway. Many applications have been found for these devices in coronary arteries in particular. Coronary arteries are prone to blockage by plaque buildup within them. A common method to treat this condition is expansion of the plaque with a balloon catheter, followed by insertion of a stent that keeps the plaque compressed thus expanding the opening through the arterial segment.

Unfortunately, a process called intimal hyperplasia sometimes occurs in which smooth muscle cells proliferate and form a new blockage within the stent. A variety of methods have been developed in an attempt to arrest his process. For instance, Fischell et. al., in U.S. Pat. No. 5,059,166, describe a method for preventing restenosis by fabricating the stent from a radioactive material that emits Beta radiation which is known to reduce intimal hyperplasia. An anti-thrombogenic coating is also placed on the outer surface of the radioisotope stent in order to reduce the incidence of acute thrombus formation at the stent location, a problem that occurs in about 2 percent of stent deployments. Human testing has shown that this device does prevent restenosis through the main body of the stent but does not do so at the ends, (a condition termed the “bow tie” effect). A serious problem with this technique is the difficulty in shipping and storing radioactive implants.

Researchers have developed many different coatings for prosthetic implants. Some are intended to reduce common problems that occur at the implant site such as infection and thrombosis. However, it is difficult to provide an implant device with coatings that address all of the potential problems that may occur, so most devices are limited to one coating addressing the problem most likely to occur. For example, in order to reduce corrosion and improve biocompatibility, corrosion resistant coatings are commonly used and include the use of inert metals such as gold and platinum and ceramic coatings such as titanium oxide, titanium nitride or calcium phosphate.

Recently, the use of antibody-antigen binding has been utilized to attract medical agents to cells and even implant devices. This is accomplished by attaching a quantity of a specific antigen to the surface of the implant. After implantation, an antibody that will bind to the surface antigen is injected into the patient. The antibody also has a medical agent bound to it. The medical agent is thereby immobilized at the implant when the antibody binds to the antigen.

There are significant limitations to this antibody-antigen system in that the antibodies are limited in the medical agent that they carry. Also, the antibodies must be administered shortly after insertion of the implant device in order to access the antigen before it is neutralized, destroyed or encapsulated by the body's immune systems.

A variety of devices other than stents can also be placed in a patient's body including such things as heart valves, artificial joints, pacemakers vascular access devices, internal fixation devices, breast implants, vascular grafts, ostomy device, bracheotherapy devices, cochlear implants, vena cava filters, ventriculo-peritoneal shunts, and pumps. All of these devices suffer from various problems to some degree including rejection, encapsulation, infection, thrombus formation and other problems.

Orthopedic implants are particularly at risk for developing infections that are difficult to treat. Infections within bone, termed osteomyelitis, is due to the poor blood flow within bone. Methods for treating infections include a systemic administration of antibiotic agents either by intra-arterial or intravenous injection. However, the high concentrations of antibiotics often needed to treat the infection often cause toxic effects in other areas of the body.

A novel device for treating osteomyelitis is disclosed in U.S. Pat. No. 3,882,858, to Klemm. He describes non-biodegradable polymer beads, that are impregnated with a topical antibiotic, and then implanted in the vicinity of the osteomyelitis. Biodegradable polymers have obvious advantages carriers for medical agents. U.S. Pat. No. 5,879,713 describes a number of biodegradable polymers known in the art and discloses delivery of bioactive molecules encoding a protein by immobilization of the bioactive molecule in a biodegradable, polymeric material adjacent to cells where delivery is desired.

The use of magnetic particles has also been investigated as a means to deliver medical agents to specific tissue areas. Many different types of magnetically sensitive particles and substrate materials have been developed for this purpose. The magnetic particle(s) and the medical agent are attached to a suitable substrate such as biodegradeable polymers, liposomes, biological cells such as red blood cells and a variety of naturally occurring organic materials such as polysaccharides, proteins, polyhyaluronic acid, hydrogels and the like.

Micro particles have an advantage over antibodies as carriers in that they can be formulated to carry almost any type of medical agent and in larger quantities. Usually a permanent magnet is used to concentrate the magnetic particles carrying a medical agent in the capillary bed of the desired tissue.

In U.S. Pat. No. 4,345,588, Widder et al. describes magnetically-localized biodegradable microspheres containing a therapeutic agent. The microspheres are injected in an artery upstream of a target capillary bed and then migrate with flow of blood to the target site and are localized there for a period of time. This concentrates the effect of the agent in the vicinity of the target capillary bed. However, when the magnetic field is removed, the microspheres and any remaining therapeutic agent leave the area and are lost. This patent does not describe a device or method that provides retention of the magnetic microspheres at a location that is deep within a patient or specific to an implant location and only provides a method to target a capillary bed. No way is provided to target a functional implant within a larger vessel or passageway. Nor can this method be adapted to administration methods such as oral or intravenous delivery.

There are many other situations where it would be desirable to direct a concentrated amount of a therapeutic and diagnostic agent to a functional implant within a body passageway. These agents include steroids, anti-inflammatory agents, anti-thrombotic drugs, gene therapy agents, chemotherapy agents and radiation therapy agents.

In U.S. Pat. No. 5,921,244, Chen et. al., describes a system and method for concentrating a medicinal substance within a patient's body, comprising the steps of providing a magnet, providing a fluid that is attracted to the magnet and includes the medicinal substance, transcutaneously inserting the magnet into the body, advancing the magnet to the internal treatment site and supplying the fluid to the patient's body so as to encourage the fluid to be conveyed to the internal treatment site to provide a substantially increased concentration of the medicinal substance at the internal treatment site.

Although the Chen et al. system provides a good way to treat some types of diseases (such as cancer tumors) it requires an undesirable transcutaneous procedure of placing the magnet directly into a tissue bed with the attendant risks of that procedure. The patent does not disclose a magnetic functional implant device intended to perform any other function in the diagnosis or treatment of a disease or disorder. Nor does the patent disclose a magnetic functional implant placed within a duct, vessel or passageway of a patient, or a way to utilize the properties of a superparamagnetic material to create a concentrated magnetic field within a patient. In one embodiment of chen et al., an electromagnet is used, which requires wires from the field coil to penetrate through the skin to get to the power source. Additionally, this patent only discloses a method of injecting the fluid and magnetic particle mixture into an artery upstream of the treatment area, resulting in accumulation of particles only within the capillary vessels downstream from the injection site. Furthermore, the patent does not disclose any way to concentrate magnetically sensitive carriers within a vein, heart chamber, lymph duct, bile duct or other passageway.

In Russian Patent No. 92001603/14, entitled, “HEART VALVE BIOPROSTHESIS,” to Kuznetsov et. al., discloses a heart valve, the valve framework of which is made of a ferromagnetic material called permendur. Magnetic pharmaceuticals can be localized in the area of the framework under the influence of a magnetic field generated by a permanent magnet placed near the valve. In another Russian patent, No. 92009305/14, entitled, “BLOOD VESSEL BIOPROSTHESIS,” Kuznetsov et. al. discloses an artificial vascular implant that has a ferromagnetic permendur wire wrapped around it. As before, magnetic pharmaceuticals can be localized in the area of the wire under the influence of a magnetic field generated by a permanent magnet placed near the vascular implant. Both of these patents are described in more detail by: Makhmudov, Sanat. Ya. et al. “Magnetically Guided Drug Transport for the Prophylaxis of Pathological Conditions and the Protection of Implants” Scientific and Clinical Applications of Magnetic Carriers Eds. Urs Häfeli, Wolfgang Sch\acute{\acute{u}}tt, Joachim Teller and Maciej Zborowski. New York: Plenum Press, 1997. 495-499.

In both Russian patents, magnetic particles were observed flowing through a vessel in which an implant was placed. There was no discussion or any indication of consideration of particles that flow through capillaries surrounding the implant. Thus, a very significant mechanism for concentration of magnetic carriers is not even considered. Additionally, both of these patents rely solely on retention of magnetic particles by the application of a magnetic field supplied by a permanent magnet external to the patient.

The Russian patents also do not disclose, teach ore suggest placement of an implant that comprises a magnet, placement of an implant that comprises a superparamagnetic the implantation of a non-magnetized ferromagnetic material that is magnetized after placement, (with the magnetizing means then removed), magnetic implants that are not placed within a blood vessel, systems or methods that allow release of the magnetic particles by demagnetizing a ferromagnetic material or removing the magnetic field placed in a superparamagnetic material. Neither of these patents contemplate or discuss the use of a superelastic ferromagnetic material.

Thus, there remains a need for a simple system and method to deliver a variety of agents to a functional implant within a patient.

### SUMMARY OF THE INVENTION

The present invention provides systems for delivering medical agents to devices implanted within a patient. Methods of using these systems are also provided.

In particular, the present invention relates to systems and methods for delivering therapeutic and diagnostic agents through a passageway to virtually any functional implant location within a patient's body.

In one aspect of the present invention, a functional implant includes a magnetized material. Such a material may be a ferromagnetic material.

In another aspect of the present invention, a functional implant includes a superparamagnetic material which can be instantaneously magnetized by application of a magnetic field near the material, and instantaneously demagnetized by removal of the same magnetic field.

In another aspect of the present invention, a functional implant includes a magnetized ferromagnetic material, where the magnetized ferromagnetic material is demagnetized by application of a degaussing device.

In another aspect of the present invention, a system for delivering a medical agent to a functional implant within a target tissue of an organism includes a magnetized functional implant disposed in a target tissue of an organism, the implant having a magnetic field, and a medical agent carried by a magnetically sensitive carrier. The carrier is introduced into a blood flow of the organism upstream from the target tissue and the carrier and medical agent migrate via the blood flow to the target tissue. After arrival, the carrier and medical agent remain substantially localized around the implant as a result of the magnetic field.

In another aspect of the present invention, a system of delivering a medicinal agent to a functional implant includes a ferromagnetic functional implant positioned in a target tissue of an organism, magnetizing means for magnetizing the implant and producing a localized magnetic field surrounding the implant, where the implant remains magnetized after removal of the magnetizing means, and introducing means for introducing a medical agent via a magnetically sensitive carrier into a blood flow of the organism upstream from the target tissue. The carrier and medical agent migrate via the blood flow to the target tissue and remain substantially localized around the target tissue as a result of the magnetic field.

In yet another aspect of the present invention, a system for delivering a medical agent to a functional implant includes a functional implant comprising a super-paramagnetic material, means for generating a magnetic field through the super-paramagnetic material, and a medical agent ferried by a magnetically-sensitive carrier.

In still yet another aspect of the present invention, a method of delivering a medical agent to a functional implant includes disposing a magnetized functional implant within a target tissue of an organism, the magnetized implant producing a magnetic field, and introducing a medical agent via a magnetically sensitive carrier into a blood flow of the organism upstream from the target tissue. The carrier migrates via the blood flow to the target tissue and the medical agent remains substantially localized as a result of the magnetic field.

In another aspect of the present invention, a method of delivering a medical agent to a functional implant, the implant including a ferromagnetic material, the method includes disposing the implant within a target tissue of an organism, magnetizing the implant to create a magnetic field surrounding the implant, and introducing a medical agent via a magnetically sensitive carrier into a blood flow of the organism upstream from the target tissue, wherein the carrier migrates via the blood flow to the target tissue and remains substantially localized at the target tissue as a result of the magnetic field.

In still yet another aspect of the present invention, a method of delivering a medical agent to a functional implant, the implant including a ferromagnetic material, the method includes disposing the implant within a target tissue of an organism, magnetizing the implant to create a magnetic field surrounding the implant, introducing a medical agent via a magnetically sensitive carrier into a blood flow of the organism upstream from the target tissue, wherein the carrier migrates via the blood flow to the target tissue and remains substantially localized at the target tissue as a result of the magnetic field, and de-magnetizing the implant so that the carrier is released from the target tissue.

In the previous aspects, the magnetically sensitive carrier is introduced either intravenously, intra-arterially, orally, intramuscularly, and/or transmucosually.

The above aspects of the present invention will become more clear with reference to the following drawings and detailed written description.

### BRIEF DESCRIPTION OF THE DRAWINGS

FIGS. 1-1A are cutaway side views of a blood vessel containing a stent implant, means to magnetize the stent implant and magnetically sensitive carriers containing a medical agent.

FIGS. 2-2A are partial cutaway side views of a magnetic bone screw in a tibia bone, magnetically sensitive carriers containing a medical agent and a means to demagnetize the bone screw.

FIG. 3 is a partial cutaway side view drawing showing a bone screw in a tibia bone and magnetically sensitive carriers containing a medical agent and a means to magnetize the bone screw.

FIG. 4 is a partial cutaway side view of a blood vessel containing a ferromagnetic superelastic alloy stent implant and magnetically sensitive carriers containing a medical agent.

FIG. 5 is a partial cutaway side view drawing showing a superparamagnetic bone screw in a tibia bone, magnetically sensitive carriers containing a medical agent and a means to concentrate a magnetic field in the bone screw.

### DETAILED DESCRIPTION OF THE INVENTION

Definitions

As used herein, a “functional implant” refers to any device that is placed in a patient's body and which has a primary function, such as the diagnosis or treatment of a disease or disorder, that does not inherently require magnetism for its use. Examples, without limitation, of such functional implants are stents, heart valves, artificial joints, pacemakers vascular access devices, orthopedic appliances such as artificial joints, internal fixation devices, screws and spinal cages. Other functional implants include breast enlargement prostheses, artificial teeth, vascular grafts, ostomy devices, bracheotherapy devices, cochlear implants, vena cava filters, sutures, ventriculo-peritoneal shunts, and pumps.

As used herein, a “therapeutic agent” refers to any substance or combination of substances used in the treatment of a disease or disorder. Examples, without limitation, of therapeutic agents are gene therapy agents, antibiotics, antineoplastics, hormones, proteins, peptides, lectins, antibodies, antivirals, radiation (via radiation sources such as cobalt, radium, yttrium, radioactive sodium iodide, etc.), anticoagulants, enzymes, hepatoprotectants, vasodilators and the like. Any therapeutic agent that can be adhered to the surface of a carrier or impregnated into the carrier or into a second material that is itself adhered to the surface of a carrier may be administered using the devices and methods herein.

As used herein, a “diagnostic agent” refers to any substance that is used to determine the nature of a disease or disorder. Examples, without limitation, of diagnostic agents are dyes that react with metabolic products of a particular disease and radioactive materials that bind to and thereby indicate the presence of disease-causing entities within a patient's body. As is the case with therapeutic agents, any diagnostic agent that can be adhered to the surface of a carrier or impregnated into the carrier or into a second material that is itself adhered to the surface of a carrier may be employed using the devices and methods herein.

As used herein an “imaging agent” shall mean a composition capable of generating a detectable image upon binding with a target and shall include radionuclides (e.g.In-111, Tc-99m, I-123, I-125 F-18, Ga-67, Ga-68, and for Positron Emission Tomography (PET) and Single Photon Emission Tomography (SPECT), unpaired spin atoms and free radicals (e.g. Fe, lanthides and Gd) and contrast agents (e.g. chelated (DTPA) manganese) for Magnetic Resonance Imaging (MRI).

As used herein, a “medical agent” refers to a therapeutic, imaging or diagnostic agent. As used herein “magnet” refers to any substance that produces a net magnetic field outside of the substance.

As used herein “magnetic field” refers to a region around a magnetized object, a moving charge, or a wire carrying electric in which objects are affected by a magnetic force.

As used herein “magnetic flux” or merely “flux” refers to the presence of a force field in a specified physical medium, or the flow of energy through a surface.

As used herein “magnetically sensitive” refers to any material that responds to a magnetic field by being either attracted to or repelled from it.

As used herein “superparamagnetic” refers to a material that is magnetized when exposed to a magnetic field but retains little or no magnetism when the magnetic field is removed.

As used herein “ferromagnetic” refers to a material that is magnetized when subjected to a magnetic field and retains magnetism when the field is removed. Substances such as iron, nickel, or cobalt, several rare earth elements and alloys of these materials exhibit ferromagnetic characteristics. As used herein “superelastic” or “pseudoplastic” refers to a material that exhibits extraordinary flexibility and torqueability. Such materials have the ability to absorb large amounts of strain energy and release it as the applied strain is removed. Superelastic materials provide nearly a constant force over a large strain range.

As used herein “demagnetizing” or “demagnetization” refers to a process of removing the magnetic field generated by a magnet. One method, termed degaussing, is to place an alternating electromagnetic field around the magnet and then gradually reducing the current of the field coil to zero.

As used herein, a “carrier” refers to at least one device, material or assembly that can be used to transport a medical agent to a target site in a patient's body. Examples, without limitation include organic particles, inorganic particles, liposomes, biological cells, virus, bacteria, prions, antibodies, antigens, hydrogels, polymers, dendrimers, nanocapsules consisting of a biodegradeable polymer shell surrounding a lipid core and the like. They may be of any suitable size ranging from 0.01 microns to about 1000 microns. They may be solid, gel or even liquid such as ferrofluids or stabilized emulsions of hydrocarbon or silicone oils.

Discussion

The present invention relates to a device and a method for localizing medical agents at the site of an implanted functional device. The invention is described below with reference to the attached drawings. However, it is to be understood that this invention is not to be construed as being in any manner limited to the embodiments in those drawings. That is, variations on the described devices and methods as well as other applications for the devices and methods will become apparent to those skilled in the art based on the disclosures herein. All such variations and applications are within the scope of this invention.

The use of permanent implants has continued to expand as new materials and surgical techniques are incorporated into medical care. One such implant is the stent. This device consists of a cylindrical metal or plastic tube containing slots or holes. Stents are placed within body passageways, usually arteries, to prop them open when weakened by disease or surgical procedures. The stent is inserted into the artery on the end of a catheter. The slots allow the stent to expand and hold the artery open. Expansion is usually accomplished either by inflation of a balloon on the catheter or with a design called a self expanding stent. Self expanding stents are made from superelastic materials such as Ni—Ti alloys. Self expanding stents are retained in a compressed, deformed state and then allowed to expand and return to their equilibrium state once they are properly positioned within the vessel.

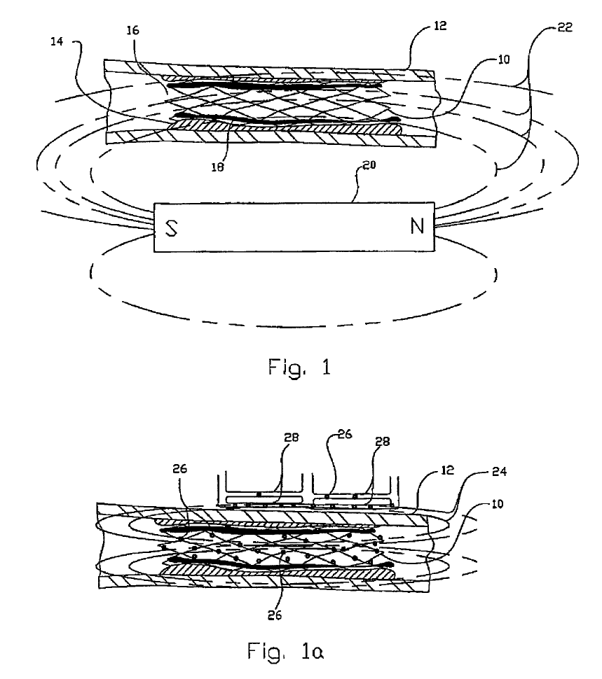

Referring to FIG. 1, a balloon expandable stent 10 is shown positioned within an artery 12 which is typically the carotid artery. Between the artery 12 and the stent 10 is plaque 14, which has been compressed by the stent 10 thereby providing a larger passageway 16 for blood flow. Smooth muscle cells have started to form a new blockage 18 within stent 10, a process called intimal hyperplasia. Stent 10 can be fabricated from a number of ferromagnetic materials such as, without limitation, 430L ferritic stainless steel. By placing an external magnet 20 near stent 10, lines of magnetic flux 22 flow through the area and the stent 10 becomes magnetized. Various types of magnets or combinations of magnets are suitable for providing the external magnetic field. One such magnet would be a gapped toroid magnet as described by Hastings in U.S. Pat. No. 6,148,823. Alternately, an electromagnet may be utilized to generate magnetic flux 22. After magnetizing stent 10, external magnet 20 may be removed.

Referring to FIG. 1a, stent 10 is now magnetized and produces lines of magnetic flux 24. Magnetically sensitive carriers 26 contain superparamagnetic iron oxide nanoparticles, agarose substrate material and therapeutic agent taxol. Taxol is a potent anticancer drug that also prevents proliferation of smooth muscle cells. It has side effects but they can be minimized by localized administration of the drug. Carriers 26 are suspended in a biocompatible fluid such as, without limitation normal saline, phosphate buffered saline, Ringer's lactate and 5% dextran in water. Optimally, carriers 26 should be as large as possible to maximize their affinity for the magnetic field generated by magnetized stent 10 and, thereby, their retention therein.

After intravenous injection, carriers 26 will make several passes through the patient's circulatory system while being accumulated by magnetic flux 22 so they must be smaller than about 5 microns in order to pass through the capillaries connecting the arterial and venous circulatory networks. Carriers 26 will also accumulate in capillary vessels 28 that are in proximity to stent 10 and contribute to the delivery of the therapeutic agent to stent 10. Large elastic blood vessels have walls that are so thick that they need their own blood supply which is provided by capillaries called the vaso vasorum. This invention would be particularly useful in these blood vessels. Because the flow in capillary vessels 28 is slow compared the flow in artery 12, the carriers are more easily retained therein by magnetic flux 24. In this manner, functional implants that are not implanted in a blood vessel may also be used to localize medical agents.

Alternately, carriers 26 may be injected into artery 12 or an artery upstream of artery 12. This provides more immediate localization of carriers 26. Various other suitable administration routes include oral, subcutaneous, intramuscular, transmucosal and the like.

After release of the taxol therapeutic agent into the smooth muscle cells, carriers 26 may be released into the blood stream by demagnetizing stent 10. This is accomplished by a process called degaussing. An alternating electromagnetic field generated by an external degaussing device is placed around stent 10. The alternating electromagnetic field is gradually brought down to zero by gradually reducing the alternating current to zero. The released carriers 26 can then be cleared from circulation by the patient's reticuloendothelial system (RES). If desired, additional carriers may be targeted to stent 10 to deliver the same or different medical agent.

The embodiment of the invention shown in FIG. 1 is particularly suitable for location in vessels that are close to the surface of a patient's body, such as the carotid artery. In this situation, external magnetic fields may be positioned in very close proximity to the functional implant. Implants placed more deeply into a patient's body may require an internal magnet or the use of a more powerful external magnet.

Another embodiment of the invention comprises an orthopedic appliance since it is often desirable to deliver a variety of medical agents to the site of an implanted orthopedic appliance. Some agents are useful in accelerating the healing of the bone while others are used to prevent or treat infections in the region of the implant. Treatment of such infections is often quite difficult due to low blood flow into bone tissues. In anterior cruciate ligament (ACL) reconstruction, a screw is used to retain the ends of a graft, such as a patellar tendon graft, tightly in position. The screws are placed adjacent to the graft in a tunnel previously drilled into the tibia and femur bones. They are wedged tightly against the graft preventing it from moving once the graft is properly positioned.

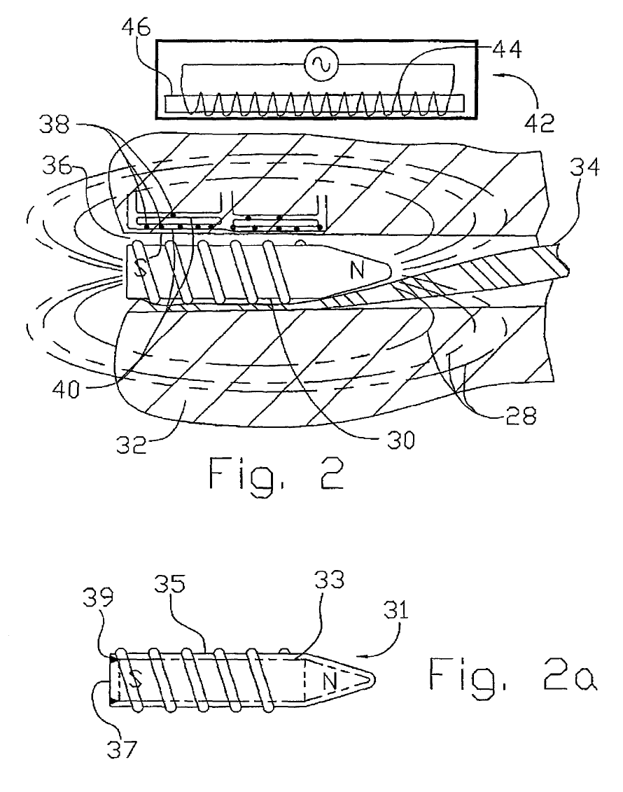

Accordingly, referring to FIG. 2, an embodiment of the present invention comprises a bone screw 30. Screw 30 is fabricated from high strength ferromagnetic alloy such as Pt—Fe—Nb. This alloy has a low corrosion rate similar to 316 stainless steel. An alternate material for screw 30 may be martensitic stainless steel alloy 410. Maximum biocompatibility of either alloy may be obtained by coating screw 30 with an appropriate coating such as TiN applied by ionized plasma deposition. Other suitable coating methods include vapor deposition and electroplating. Many other types of metal, polymer and ceramic coatings suitable for coating the implant are known in the art.

Screw 30 is placed in tibia 32 and retains graft 34 in hole 36. The screw is magnetized before being positioned in the bone. In order to increase the rate of new bone formation around screw 30 and into hole 36, magnetically-sensitive carriers 38 containing bone morphogenic protein (BMP) are administered to the patient by intravenous injection. Normally BMP would have a very short half life in circulating blood. However, encapsulating the BMP in a carrier 38 protects it from degradation. A carrier such as a magnetoliposome can achieve an extended circulating time by grafting polyethyleneglycol (PEG) to the carrier. This reduces carrier recognition by circulating macrophages. Other configurations and additives, known in the art also increase the capability of a carrier to achieve a ‘stealth’ configuration.

The carriers are distributed throughout the patient's vascular system (not shown) and collect in capillary vessels 40 next to screw 30 by the force of magnetic flux 28 from screw 30. After a period of time to allow uptake of BMP, the carriers 38 may be released from capillaries 40 by de-magnetizing screw 30. This is accomplished by placing a degaussing device 42 in proximity to screw 30. The degaussing device provides an alternating electromagnetic field around screw 30. Degaussing device 42 contains electromagnetic field coil 44 wrapped around core 46. The alternating electromagnetic field is gradually brought down to zero by gradually reducing the alternating current through field coil 44 to zero. This eliminates the magnetism of screw 30 and releases carriers 38 into the vascular system where they are eventually cleared by the RES.

Rare earth ferromagnetic materials are also excellent choices for bone applications but must be encased in a shell of a high strength, biocompatible material. One such assembly is shown in FIG. 2a. In this embodiment, bone screw 31 is composed of a ferromagnetic core 33, made from SmCo and a two piece casing consisting of housing 35 and cap 37. The casing parts are made from titanium or other high strength, biocompatible material. The device is assembled by inserting the core 33 into housing 35 and welding the cap 37 to housing 35 at weld joint 39.

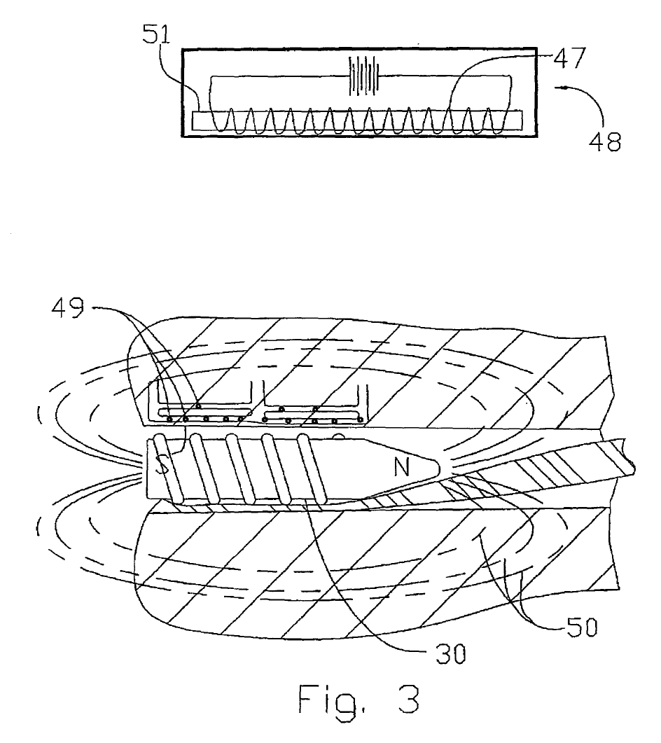

If the tissue around the screw becomes infected, an additional therapeutic agent such as an antibiotic, carried by a magnetically sensitive carrier may be delivered to the site by re-magnetizing the screw and proceeding as above. Thus (referring to FIG. 3), a magnetic field is generated around screw 30 by a suitable magnetizing means 48 and screw 30 is re-magnetized. Magnetizing means 48 is an electromagnet containing core 51 and field winding 47. After re-magnetizing screw 30, magnetizing means 48 is removed leaving screw 30 with its own magnetic flux 50. Additional magnetically-sensitive carriers 49 containing an antibiotic may be delivered and held by magnetic flux 50 from screw 30 as previously described. Permanent magnets such as a gappe3d toroid magnet (not shown) may also be used to re-magnetize screw 30. Stronger fields, able to penetrate deeper into tissue, can be generated by an electromagnet.

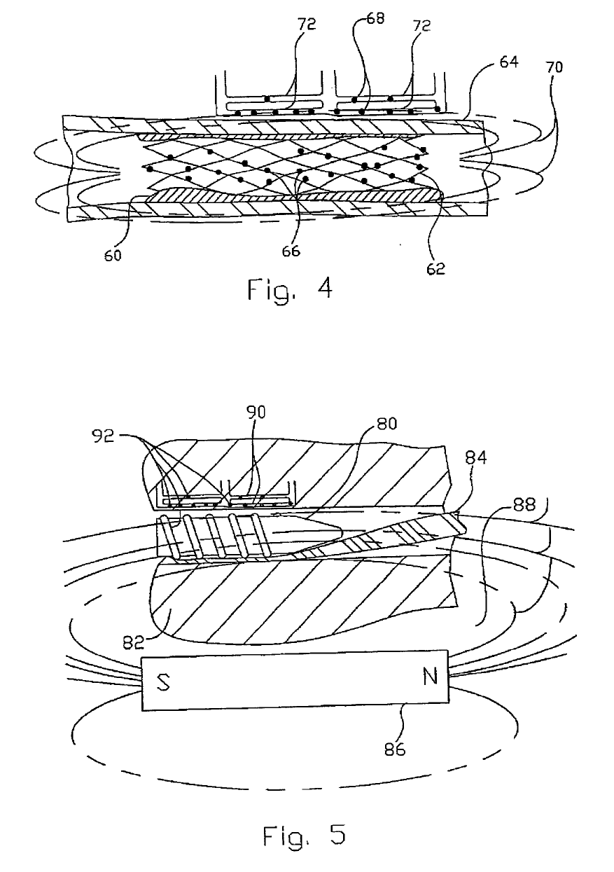

FIG. 4 illustrates another embodiment of the invention. Where self expanding stent 62 is placed within a coronary artery 64 at the site of a previously dilated plaque 60. Fabricated from a ferromagnetic superelastic alloy such as Ni₂MnGa, Ni₂MnGa, FePd and FeNiCoTi, stent 62 is magnetic at the time of implantation, (although it may also be implanted in a nonmagnetic condition and magnetized by temporary application of an electromagnetic or magnetic field) and may be covered with a biocompatible material such as polyurethane resin. Magnetically-sensitive carriers 66, contains nitric oxide, a substance known to reduce neointimal hyperplasia, is administered into the patient and collect on the surface of stent 62. Additional carriers 66 collect in capillaries 72 in tissue adjacent to stent 62, reducing the possibility of restenosis.

FIG. 5 again illustrates a bone screw 80, placed in tibia 82 for retaining graft 84. Screw 80 is fabricated by metal injection molding using a mixture of 316L stainless steel and superparamagnetic iron oxide, metal injection molded parts may be made with a biocompatible stainless steel powder such as IMET 316L. The finished part is corrosion resistant and includes high magnetic permeability due to the presence of the superparamagnetic particles. By placing a permanent magnet 86 near screw 80, lines of magnetic flux 88 flow through the area and the superparamagnetic particles within screw 80 concentrate the magnetic flux 88 closely around screw 80 in capillaries 90.

Various types of magnets, combinations of magnets or electromagnets are suitable for providing the magnetic field. Removing magnet 86 removes magnetic flux 88 and because screw 80 contains a superparamagnetic material and non-magnetic material, thus, screw 80 does not retain any magnetism. Magnetically sensitive carriers 92 containing a desired medical agent are concentrated around screw 80. If magnetically sensitive carriers 92 incorporate superparamagnetic particles, they will not retain any magnetism. They will be released from the site and carried away by normal blood flow when magnet 86 is removed. Carriers of superparamagnetic iron oxide particles with a silane coating approximately 1 micron in size may be obtained from Polysciences Europe GmbH. Another source of spherical carriers is Vector Laboratories who distributes magnetically sensitive carriers that are available in a variety of materials such as a mixture of magnetite and cellulose. Other implantable devices may be made from composites such as molded polymers or ceramics containing superparamagnetic particles. Imbedding the superparamagnetic particles in structures such as glass beads before mixing with the polymer will improve handling and maintain separation of the superparamagnetic particles.

Having presented the present invention in view of the above described embodiments, various alterations, modifications, and improvements are intended to be within the scope and spirit of the invention. The foregoing description is by way of example only and is not intended as limiting. The invention's limit is defined only in the following claims and the equivalents thereto.

What is claimed is:

1. A functional implant comprising a magnetized material.

2. The functional implant according to claim 1, wherein said material comprises a ferromagnetic material.

3. A functional implant comprising a superparamagnetic material.

4. The functional implant according to claim 1 or 2, wherein the implant includes a shape memory material.

5. The functional implant according to claim 2, wherein the ferromagnetic material comprises a superelastic ferromagnetic material that is selected from the group comprising an alloy of: Ni₂MnGa, FePd or FeNiCoTi.

6. The implant according to claim 1 or 3, wherein the functional implant comprises any one of a stent, an artificial heart valve, an orthopedic appliance, a surgical staple, a pacemaker, a pump, a vascular graft, a vascular access device, an artificial tooth, an ostomy device, a breast enlargement prosthesis, a bracheotherapy device, a cochlear implant, a vascular filter, a suture, and a ventriculo-peritoneal shunt.

7. The functional implant according to claim 1 or 3, further comprising a biocompatible coating.

8. The functional implant according to claim 3, wherein the functional implant comprises a matrix of a biocompatible metal and a superparamagnetic material.

9. The functional implant according to claim 3, wherein the functional implant comprises a matrix of a biocompatible polymer and a superparamagnetic material.

10. The functional implant according to claim 3, wherein the functional implant comprises a matrix of a biocompatible ceramic and a superparamagnetic material.

11. A functional implant comprising a ferromagnetic material magnetized by application of an external magnetic field, wherein the functional implant remains magnetized after removal of the external magnetic field.

12. A functional implant comprising a magnetized ferromagnetic material, wherein the magnetized ferromagnetic material is demagnetized by application of a degaussing device.

13. A system for delivering a medical agent to a functional implant within a target tissue of an organism comprising:
a magnetized functional implant disposed in a target tissue of an organism, the implant having a magnetic field; and
a medical agent carried by a magnetically sensitive carrier, wherein
the carrier is introduced into a blood flow of the organism upstream from the target tissue,
the carrier and medical agent migrate via the blood flow to the target tissue, and
the carrier and medical agent remain substantially localized around the implant as a result of the magnetic field.

14. A system of delivering a medicinal agent to a functional implant comprising:
a ferromagnetic functional implant positioned in a target tissue of an organism;
magnetizing means for magnetizing the implant and producing a localized magnetic field surrounding the implant, wherein the implant remains magnetized after removal of the magnetizing means; and
introducing means for introducing a medical agent via a magnetically sensitive carrier into a blood flow of the organism upstream from the target tissue, wherein the carrier and medical agent migrate via the blood flow to the target tissue and remain substantially localized around the target tissue as a result of the magnetic field.

15. The system according to claim 14, further comprising a demagnetizing means for demagnetizing the implant.

16. The system according to claim 15, wherein said demagnetizing means comprises a degaussing device.

17. A system for delivering a medical agent to a functional implant comprising:
a functional implant comprising a super-paramagnetic material;
means for generating a magnetic field through the super-paramagnetic material;
a medical agent ferried by a magnetically-sensitive carrier.

18. A method of delivering a medical agent to a functional implant comprising:
disposing a magnetized functional implant within a target tissue of an organism, the magnetized implant producing a magnetic field; and
introducing a medical agent via a magnetically sensitive carrier into a blood flow of the organism upstream from the target tissue, wherein the carrier migrates via the blood flow to the target tissue and wherein the medical agent remains substantially localized as a result of the magnetic field.

19. A method of delivering a medical agent to a functional implant, the implant comprising a ferromagnetic material, the method comprising:
disposing the implant within a target tissue of an organism;
magnetizing the implant to create a magnetic field surrounding the implant;
introducing a medical agent via a magnetically sensitive carrier into a blood flow of the organism upstream from the target tissue, wherein the carrier migrates via the blood flow to the target tissue and remains substantially localized at the target tissue as a result of the magnetic field.

20. A method of delivering a medical agent to a functional implant, the implant comprising a ferromagnetic material, the method comprising:
disposing the implant within a target tissue of an organism;
magnetizing the implant to create a magnetic field surrounding the implant;
introducing a medical agent via a magnetically sensitive carrier into a blood flow of the organism upstream from the target tissue, wherein the carrier migrates via the blood flow to the target tissue and remains substantially localized at the target tissue as a result of the magnetic field; and
de-magnetizing the implant so that the carrier is released from the target tissue.

21. The method according to claim 18, 19 or 20, wherein the magnetically sensitive carrier is introduced intravenously, intra-arterially, orally, intramuscularly, and/or transmucosually.
